(19) United States
(12) Patent Application Publication
(10) Pub. No.: US 2013/0071466 A1
(43) Pub. Date: Mar. 21, 2013

(54) METHODS AND COMPOSITIONS FOR TREATING GASTRIC DISORDERS

(71) Applicants: Michael B. Chancellor, Pittsburgh, PA (US); Jonathan H. Kaufman, Pittsburgh, PA (US)

(72) Inventors: Michael B. Chancellor, Pittsburgh, PA (US); Jonathan H. Kaufman, Pittsburgh, PA (US)

(73) Assignee: Lipella Pharmaceuticals Inc.

(21) Appl. No.: 13/368,186

(22) Filed: Feb. 7, 2012

Related U.S. Application Data
(63) Continuation-in-part of application No. 12/651,075, filed on Dec. 31, 2009.
Application No. 12/651,075 is a continuation-in-part of application No. 11/546,025, filed on Oct. 11, 2006, now Pat. No. 8,110,217.
Application No. 11/546,025 is a division of application No. 10/218,797, filed on Aug. 13, 2002, now Pat. No. 7,063,860.
(60) Provisional application No. 60/725,402, filed on Oct. 11, 2005.
(60) Provisional application No. 60/311,868, filed on Aug. 13, 2001.

(51) Int. Cl. A61K 9/127; A61P 1/00; A61P 1/04; A61P 31/00; A61P 29/00; A61K 35/74; A61P 35/00

(52) U.S. Cl. 424/450; 424/780

(57) ABSTRACT

Improved efficacy in treatment of gastric disorders with botulinum toxin is obtained using liposomal encapsulated botulinum formulations for topical administration of the botulinum toxin. The liposomes are typically administered in a physiologically acceptable carrier such as saline or phosphate buffered saline by application onto the surface of the tissue within into the gastrointestinal (GI) tract in need of treatment. Preferably, the formulation is applied via a roller, sponge or nozzle that is attached to an endoscope.

### CROSS-REFERENCE TO RELATED APPLICATIONS

This application is a continuation-in-part of U.S. Ser. No. 12/651,075 filed Dec. 31, 2009, which claims priority to U.S. Ser. No. 11/546,025 filed Oct. 11, 2006, now U.S. Pat. No. 8,110,217, which claims priority to U.S. Ser. No. 60/701,431 filed Jul. 20, 2005 and U.S. Ser. No. 60/725,402 filed Oct. 11, 2005, and which is a divisional of U.S. Ser. No. 10/218,797 filed Aug. 13, 2002, now U.S. Pat. No. 7,063,860, which claims priority to U.S. Ser. No. 60/311,868 filed Aug. 13, 2001.

### FIELD OF THE INVENTION

The present invention is generally in the field of to compositions, kits and methods for administering liposomal encapsulated botulinum toxin to treat gastric disorders.

### BACKGROUND OF THE INVENTION

Millions of people suffer from gastric disorders such as gastroesophageal reflux disease (GERD), achalasia, Crohn's disease, diverticulosis, diverticulitis, gallstones, hiatal hernia, heartburn, gastric stasis, pyloric valve (or other sphincter in the gastrointestinal tract) malfunction or spasm, H. pylori induced ulcers, peptic ulcers, irritable bowel syndrome, stomach ulcers, esophageal spasm, duodenal ulcers, colitis, ulcerative colitis and lactose and gluten intolerances. The literature shows that many gastric disorders or diseases can be treated by injecting botulinum toxin (BoNT) into the esophagus region.

Gastroesophageal reflux disease (also called peptic esophagitis and reflux esophagitis) is an inflammation of the esophagus resulting from regurgitation of gastric contents into the esophagus. Some gastroesophageal reflux is a normal condition that often occurs without symptoms after meals. However, the reflux can become a serious problem when it is due to an incompetent (weakened) lower esophageal sphincter, a band of muscle fibers that closes off the esophagus from the stomach. When this occurs, acidic or alkaline gastric contents from the stomach can return to the esophagus through the lower esophageal sphincter and cause the symptoms of GERD. Conditions which can cause an incompetent esophageal sphincter with resulting GERD include pregnancy, hiatal hernia, obesity, recurrent or persistent vomiting, and nasogastric tubes. GERD is also a risk factor after esophageal surgery and esophageal stricture.

The symptoms of GERD include heartburn, belching, regurgitation of food, nausea, vomiting, hoarseness of voice, sore throat, difficulty swallowing, chest pain, and cough. Diagnostic tests for GERD include a stool guaiac test, continuous esophageal pH monitoring, esophageal manometry, a barium swallow, and a Bernstein test for gastric acid reflux.

Mirbagheri et al., Dig Dis. Sci. 53(10):2621-26 (2008) showed that endoscopic pyloric BoNT injection alleviates symptoms of both gastroesophageal reflux disease and gastroparesis. Eleven patients with GERD plus gastroparesis received BoNT injection. Mirbagheri reported that the injection improved both gastroparesis- and reflux-related symptoms in the majority of patients but the duration of relief was relatively short.

Kroupa et al., Dis Esophagus. 23(2):100-5 (2010) showed that injection of botulinum toxin (BoNT) and pneumatic dilatation are available methods in nonsurgical treatment of achalasia. Each patient received injection of 200 IU of BoNT into the lower esophageal sphincter (LES) during endoscopy and 8 days later pneumatic dilatation (PD) under X-ray control was performed. The study showed that BoNT had therapeutic effect but that the combined therapy of BoNT and PD was not significantly superior to PD alone.

Porter et al., Neurogastroenterol Motil., 23(2):139-44 (2011) showed that BoNT injections can provide symptom relief in dysphagia syndromes with incomplete lower esophageal sphincter relaxation (LESR).

Bashashati et al., Dis Esophagus., 23(7):554-60 (2010) showed that endoscopic injection of BoNT to the esophagus results in symptomatic benefit in patients with diffuse esophageal spasm.

In all reported cases, botulinum toxin was effective in decreasing some symptoms of gastric disorders for at least a short period of time. However, results varied and in many cases were not better than alternative treatments.

All of the current BoNT therapies for gastric disorders are performed via injections. The injection can cause pain and unwanted side effects in patients. It is therefore desirable to have an alternative BoNT therapy.

Therefore, it is an object of the invention to provide improved compositions and methods of administration of botulinum toxin to provide relief from one or more symptoms of gastric disorders.

It is a further object of the invention to provide compositions and methods of administering the compositions that provide relief from one or more symptoms of gastric disorders that are effective for a prolonged period of time compared to current therapies.

### SUMMARY OF THE INVENTION

Lipid formulations, such as liposomes, micelles, or emulsions, containing botulinum toxin (BoNT) encapsulated in the lipid formulation, preferably liposomes and a carrier, are sprayed or painted into or onto the eosophagus. The BoNT can be BoNT A-G, preferably BoNT A, C or E, more preferably BoNT A. The pharmaceutically acceptable carrier is preferably an aqueous carrier, which can be in the form of a liquid or gel. The lipids are preferably phospholipids or sphingolipids. In the liposome, the molar ratio of a phospholipid to second lipid can range from about 5:1 to about 1:1. In one embodiment, the unit dosage formulation contains a dry powder of liposomal encapsulated BoNT, which is reconstituted with a pharmaceutically acceptable carrier, preferably an aqueous carrier to form the formulations containing BoNT encapsulated in liposomes. The resulting formulation has a liposomal BoNT concentration suitable for treatment or relief from one or more symptoms of a gastric disorder and/or the gastric disorder.

The formulations can be administered to the gastrointestinal tract, preferably the esophagus, stomach, small intestine and large intestine, in an effective amount to treat a gastrointestinal disorder or provide relief from one or more symptoms of the gastrointestinal disorder. The dose administered of BoNT is from about 1 to about 200 units, preferably about 1 to about 25 units. The formulations can be administered using endoscopy. The endoscope can have an applicator that administers the formulation. Suitable applicators are spray devices, sponges, gauze and rollers. The formulation can be sprayed, painted or rolled onto a desired area in the GI tract. In a preferred embodiment, the formulation is sprayed or painted onto the eosophagus or other portions of the gastrointestinal tract, using an endoscope.

The formulations can be used to treat a variety of gastric disorders, such as GERD, achalasia, Crohn's disease, diverticulosis, diverticulitis, gallstones, hiatal hernia, gastric stasis, pyloric valve (or other G1 sphincter) malfunction or spasm, H. pylori induced ulcers, peptic ulcers, esophageal spasm, irritable bowel syndrome—including lactose and gluten intolerance, stomach ulcers, duodenal ulcers, colitis or ulcerative colitis, and are preferably effective at treating GERD or achalasia, or one or more symptoms thereof. The formulations can treat a variety of symptoms associated with gastric disorders, such as heartburn, belching, regurgitation of food, nausea, vomiting, hoarseness of voice, sore throat, difficulty swallowing, chest pain, or cough.

### DETAILED DESCRIPTION OF THE INVENTION

### I. Formulations

Lipid encapsulation increases absorption of botulinum toxin. Liposome encapsulation also protects BoNT from degradation in vivo and allows unhindered absorption across the tissue from liposomes adhering to the tissue surface. Since BoNT is entrapped inside the liposomes, it is not vulnerable to dilution by physiological secretions and localized concentration of BoNT at the liposome surface can be high enough to hasten the entry of leached BoNT from liposomes adhering to the surface of the area of administration of the gastrointestinal (GI) tract.

Botulinum toxin is a large protein (molecular weight≈150 kDa) which does not diffuse through tissue easily to reach its target. The target protein for BoNT resides in a lipid environment. Liposomes can enhance the activity of metalloproteases such as BoNT by allowing more efficient delivery of the BoNT to the tissue.

### I. Formulations

The formulations contain Botulinum toxin (BoNT) encapsulated in an liposomes, an emulsion, or micells and a carrier. Optionally, the formulations contain one or more excipients. The preferred carrier is liposomal. The formulations can be in the form of a liquid or gel, preferably as a liquid.

A. Liposomes

Liposomes are spherical vesicles, composed of concentric phospholipid bilayers separated by aqueous compartments. Liposomes adhere to and create a molecular film on cellular surfaces. (Gregoriadis, et al., Int J Pharm 300, 125-30 2005; Gregoriadis and Ryman, Biochem J 124, 58P (1971)). The lipid vesicles comprise either one or several aqueous compartments delineated by either one (unilamellar) or several (multilamellar) phospholipid bilayers (Sapra, et al., Curr Drug Deliv 2, 369-81 (2005)). The success of liposomes in the clinic has been attributed in part to the nontoxic nature of the lipids used in their formulation. Both the lipid bilayer and the aqueous interior core of liposomes can serve the purpose of treatment. Liposomes have been well studied as carrier of toxins for enhancing their efficacy at lower doses (Alam, et al., Mol Cell Biochem 112, 97-107 1992; Chaim-Matyas, et al., Biotechnol Appl Biochem 17 (Pt 1), 31-6 1993; de Paiva and Dolly, FEBS Lett 277, 171-4 (1990); Freitas and Frezard, Toxicon 35, 91-100 (1997); Mandal and Lee, Biochim Biophys Acta 1563, 7-17 (2002)).

Liposomes have been widely studied as drug carriers for a variety of chemotherapeutic agents (thousands of scientific articles have been published on the subject) (see, e.g. Gregoriadis, N Engl J Med 295, 765-70 (1976); Gregoriadis, et al., Int J Pharm 300, 125-30 (2005)). Water-soluble anticancer substances such as doxorubicin can be protected inside the aqueous compartment(s) of liposomes delimited by the phospholipid bilayer(s), whereas fat-soluble substances such as amphotericin and capsaicin can be integrated into the phospholipid bilayer (Aboul-Fadl, Curr Med Chem 12, 2193-214 (2005); Tyagi, et al,, J Urol 171, 483-9 (2004)). Topical and vitreous delivery of cyclosporine was drastically improved with liposomes (Lallemand, et al., Eur J Pharm Biopharm 56, 307-18 2003). Delivery of chemotherapeutic agents lead to improved pharmacokinetics and reduced toxicity profile (Gregoriadis, Trends Biotechnol 13, 527-37 (1995); Gregoriadis and Allison, FEBS Lett 45, 71-4 1974; Sapra, et al., Curr Drug Deliv 2, 369-81 (2005)). More than ten liposomal and lipid-based formulations have been approved by regulatory authorities and many liposomal drugs are in preclinical development or in clinical trials (Barnes, Expert Opin Pharmacother 7, 607-15 (2006); Minko, et al., Anticancer Agents Med Chem 6, 537-52 (2006)). Fraser, et al. (Urology, 2003; 61: 656-663) demonstrated that intravesical instillation of liposomes enhanced the barrier properties of dysfunctional tissue and partially reversed the high micturition frequency in a rat model of hyperactive bladder induced by breaching the uroepithelium with protamine sulfate and thereafter irritating the bladder with KCl. Tyagi et al. J Urol., 2004; 171; 483-489 reported that liposomes are a superior vehicle for the intravesical administration of capsaicin with less vehicle induced inflammation in comparison with 30% ethanol. Clinical studies have proven the efficacy of liposomes as a topical healing agent (Dausch, et al., Klin Monatsbl Augenheilkd 223, 974-83 (2006); Lee, et al., Klin Monatsbl Augenheilkd 221, 825-36 (2004)). Liposomes have also been used in ophthalmology to ameliorate keratitis, corneal transplant rejection, uveitis, endophthalmitis, and proliferative vitreoretinopathy (Ebrahim, et al., Surv Ophthalmol. 50(2):167-82 (2005); Li, et al., 2007). The safety data with respect to acute, subchronic, and chronic toxicity of liposomes has been assimilated from the vast clinical experience of using liposomes in the clinic for thousands of patients. The safe use of liposomes for the intended clinical route is also supported by its widespread use as a vehicle for anticancer drugs in patients.

Emulsions and micelles can also be used, although they are not as preferred as liposomes, primarily due to stability issues. Formulations and methods of manufacture are well known.

a. Lipids

The liposomes contain one or more lipids. The lipids can be neutral, anionic or cationic lipids at physiologic pH.

Suitable neutral and anionic lipids include, but are not limited to, sterols and lipids such as cholesterol, phospholipids, lysolipids, lysophospholipids, sphingolipids or pegylated lipids. Neutral and anionic lipids include, but are not limited to, phosphatidylcholine (PC) (such as egg PC, soy PC), including, but limited to, 1,2-diacyl-glycero-3-phosphocholines; phosphatidylserine (PS), phosphatidylglycerol, phosphatidylinositol (PI); glycolipids; sphingophospholipids such as sphingomyelin and sphingoglycolipids (also known as 1-ceramidyl glucosides) such as ceramide galactopyranoside, gangliosides and cerebrosides; fatty acids, sterols, containing a carboxylic acid group for example, cholesterol; 1,2-diacyl-sn-glycero-3-phosphoethanolamine, including, but not limited to, 1,2-dioleylphosphoethanolamine (DOPE), 1,2-dihexadecylphosphoethanolamine (DHPE), 1,2-distearoylphosphatidylcholine (DSPC), 1,2-dipalmitoyl phosphatidylcholine (DPPC), and 1,2-dimyristoylphosphatidylcholine (DMPC). The lipids can also include various natural (e.g., tissue derived L-α-phosphatidyl: egg yolk, heart, brain, liver, soybean) and/or synthetic (e.g., saturated and unsaturated 1,2-diacyl-sn-glycero-3-phosphocholines, 1-acyl-2-acyl-sn-glycero-3-phosphocholines, 1,2-diheptanoyl-SN-glycero-3-phosphocholine) derivatives of the lipids. In one embodiment, the liposomes contain a phosphaditylcholine (PC) head group, and preferably sphingomyelin. In a preferred embodiment, the liposomes contain DPPC. In a preferred embodiment, the liposomes contain a neutral lipid, preferably 1,2-dioleoylphosphatidylcholine (DOPC).

In one embodiment, the formulations contain non-cationic liposomes, preferably of sphingomyelin, and a pharmaceutically acceptable carrier. In a further embodiment, the liposomes include a sphingomyelin metabolite and at least one lipid. Sphingomyelin metabolites includes, for example and without limitation ceramide, sphingosine or sphingosine 1-phosphate.

The concentration of the sphingomyelin metabolites included in the lipids used to formulate the liposomes can range from about 0.1 mol % to about 10.0 mol %, preferably from about 2.0 mol % to about 5.0 mol %, and more preferably can be in a concentration of about 1.0 mol %.

Suitable cationic lipids in the liposomes include, but are not limited to, N-[1-(2,3-dioleoyloxy)propyl]-N,N,N-trimethyl ammonium salts, also references as TAP lipids, for example methylsulfate salt. Suitable TAP lipids include, but are not limited to, DOTAP (dioleoyl-), DMTAP (dimyristoyl-), DPTAP (dipalmitoyl-), and DSTAP (distearoyl-). Suitable cationic lipids in the liposomes include, but are not limited to, dimethyldioctadecyl ammonium bromide (DDAB), 1,2-diacyloxy-3-trimethylammonium propanes, N-[1-(2,3-dioloyloxy)propyl]-N,N-dimethyl amine (DODAP), 1,2-diacyloxy-3-dimethylammonium propanes, N-[1-(2,3-dioleyloxy)propyl]-N,N,N-trimethylammonium chloride (DOTMA), 1,2-dialkyloxy-3-dimethylammonium propanes, dioctadecylamidoglycylspermine (DOGS), 3-[N-(N′,N′-dimethylamino-ethane)carbamoyl]cholesterol (DC-Chol); 2,3-dioleoyloxy-N-(2-(sperminecarboxamido)-ethyl)-N,N-dimethyl-1- propanaminium trifluoro-acetate (DOSPA), β-alanyl cholesterol cetyl trimethyl ammonium bromide (CTAB), diC₁₄-amidine, N-ferf-butyl-N′-tetradecyl-3-tetradecylamino-propionamidine, N-(alpha-trimethylammonioacetyl)didodecyl-D-glutamate chloride (TMAG), ditetradecanoyl-N-(trimethylammonio-acetyl)deithanolamine chloride 1,3-dioleoyloxy-2-(6-carboxy-spermyl)-propylamide (DOSPER), and N,N,N′,N′-tetramethyl-, N′-bis(2-hydroxylethyl)-2,3-dioleoyloxy-1,4-butanediammonium iodide. In one embodiment, the cationic lipids can be 1-[2-(acyloxy)ethyl]2-alkyl(alkenyl)-3-(2-hydroxyethyl)-imidazolinium chloride derivatives, for example, 1-[2-(9(Z)-octadecenoyloxy)ethyl]-2-(8(Z)-heptadecenyl-3-(2-hydroxyethyl)imidazolinium chloride (DOTIM), and 1-[2-(hexadecanoyloxy)ethyl]-2-pentadecyl-3-(2-hydroxyethyl)imidazolinium chloride (DPTIM). In one embodiment, the cationic lipids can be 2,3-dialkyloxypropyl quaternary ammonium compound derivatives containing a hydroxyalkyl moiety on the quaternary amine, for example, 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-dimetyl-hydroxypropyl ammonium bromide (DORIE-HP), 1,2-dioleyl-oxypropyl-3-dimethyl-hydroxybutyl ammonium bromide (DORIE-HB), 1,2-dioleyloxypropyl-3-dimethyl-hydroxypentyl ammonium bromide (DORIE-Hpe), 1,2-dimyristyloxypropyl-3-dimethyl-hydroxylethyl ammonium bromide (DMRIE), 1,2-dipalmityloxypropyl-3-dimethyl-hydroxyethyl ammonium bromide (DPRIE), and 1,2-disteryloxypropyl-3-dimethyl-hydroxyethyl ammonium bromide (DSRIE).

The lipids may be formed from a combination of more than one lipid, for example, a charged lipid may be combined with a lipid that is non-ionic or uncharged at physiological pH. Non-ionic lipids include, but are not limited to, cholesterol and DOPE (1,2-dioleolylglyceryl phosphatidylethanolamine), with cholesterol being most preferred. The molar ratio of a first phospholipid, such as 1,2-diacyl-glycero-3-phosphocholines, to second lipid can range from about 5:1 to about 1:1 or 3:1 to about 1:1, more preferably from about 1.5:1 to about 1:1, and most preferably, the molar ratio is about 1:1.

b. Liposome Core

The liposomes typically have an aqueous core. The aqueous core can contain water or a mixture of water and alcohol. Suitable alcohols include, but are not limited to, methanol, ethanol, propanol (such as isopropanol), butanol (such as n-butanol, isobutanol, sec-butanol, tert-butanol), pentanol (such as amyl alcohol, isobutyl carbinol), hexanol (such as 1-hexanol, 2-hexanol, 3-hexanol), heptanol (such as 1-heptanol, 2-heptanol, 3-heptanol and 4-heptanol) or octanol (such as 1-octanol) or a combination thereof.

c. Ratio of BoNT to Lipid

The BoNT to lipid ratio (unit of BoNT per mg of lipid) can be controlled to regulate the efficiency of the BoNT. Suitable BoNT to lipid ratios include, but are not limited to, 1:1, 1:0.9, 1:0.8, 1:0.7, 1:0.6, 1:0.5, 1:0.4, 1:0.3, 1:0.2 or 1:0.1 (unit of BoNT per mg of lipid) . In one embodiment, the BoNT to lipid ratio is 1:0.5.

B. Botulinum Toxin (BoNT) and Other Drugs

Botulinum neurotoxin (BoNT) refers to botulinum serotypes A, B, C, D, E, F, G and all modified, substituted or fragment versions of these toxins that have a blocking effect on snare proteins. These include any substitution or modification of at least 1 amino acid of a naturally produced toxin or synthetically produced toxins. These modifications can be made with recombinant techniques. Also included are toxins with removal or substitution of the binding domain and/or translocation domain. Some of these variations of BoNT types A to G are discussed in U.S. Pat. No. 7,491,799 and by Bland et al. (Protein Expr Purif., 71(1):62-73 (2010)).

Botulinum toxin is produced by Clostridium botulinum and is regarded as the most potent biological toxin known (Smith & Chancellor, J Urol, 171: 2128 (2004)). BoNT has been used effectively to treat different conditions with muscular hypercontraction. BoNT-A is the most common clinically used botulinum toxin among seven immunologically distinct neurotoxins (types A to G). BoNT-A and BoNT-B have been used successfully for the treatment of spinal cord injured patients with neurogenic bladder hyperactivity using intradetrusor BoNT-A injection at multiple sites.

BoNT is known to exert effects by inhibiting acetylcholine (“ACh”) release at the neuromuscular junction as well as autonomic neurotransmission. After intramuscular injection of BoNT, temporary chemodenervation and muscle relaxation can be achieved in skeletal muscle as well as in smooth muscle (Chuang & Chancellor, J Urol. 176(6 Pt 1):2375-82 (2006)). Smith et al. (J Urol, 169: 1896 (2003)) found that BoNT injection into the rat proximal urethral sphincter caused marked decreases in labeled norepinephrine at high but not at low electrical field stimulation, indicating that BoNT inhibits norepinephrine release at autonomic nerve terminals.

In one embodiment, the BoNT can be BoNT A-G, preferably BoNT A, C or E, more preferably BoNT A.

The formulations or liposomes optionally contain one or more drugs in place of or in addition to BoNT. These may include antiinfectives such as drugs to treat infections caused by bacteria, fungus, or viruses, analgesics, anti-inflammatories, anti-ulcer medications, antispasmodics, or other drugs used to treat gastric conditions.

C. Carriers

The formulations contain a pharmaceutically acceptable carrier, preferably a pharmaceutically acceptable aqueous carrier suitable for use in a nasal spray device. Suitable carriers include, but are not limited to, water or aqueous solutions containing pharmaceutically acceptable salts, buffers, or mixtures thereof, for example saline or phosphate buffered saline (PBS).

The concentration of liposomal BoNT in the carrier can be varied. Suitable concentrations of liposomal BoNT in the carrier include, but are not limited to, 0.05 mg/ml to 10 mg/ml, preferably 0.05 mg/ml to 5 mg/ml, more preferably 0.05 mg/ml to 2.5 mg/ml.

### II. Methods of Manufacturing

A. Manufacturing of Liposomes

Methods of manufacturing liposomes are described in the literature cited above and are well known. In one embodiment, aqueous liposome suspensions are produced by microfluidization. However, the end product may be subject to a series of stability problems such as aggregation, fusion and phospholipid hydrolysis (Nounou, et al., Acta Pol Pharm 62, 381-91 (2005)).

The liposomal product must possess adequate chemical and physical stability before its clinical benefit can be realized (Torchilin, Adv Drug Deliv Rev 58, 1532-55 (2006)). In a preferred embodiment, dehydrated liposomes are prepared using a suitable method For example, in a preferred embodiment dehydrated liposomes formed from a homogenous dispersion of phospholipid in a tent-butyl alcohol (TBA)/water cosolvent system. The isotropic monophasic solution of liposomes is freeze dried to generate dehydrated liposomal powder in a sterile vial. The freeze drying step leaves empty lipid vesicles or dehydrated liposomes after removing both water and TBA from the vial. On addition of a physiologically acceptable carrier, such as physiological saline or PBS, the lyophilized product spontaneously forms a homogenous liposome preparation (Amselem, et al., J Pharm Sci 79, 1045-52 (1990); Ozturk, et al., Adv Exp Med Biol 553, 231-42 (2004)). Liposomes having low lipid concentrations are well-suited for this method. The ratio of lipid to TBA is an important factor affecting the size and the polydispersity of resulting liposome preparation.

B. Preparation for Liposomal BoNT

In one embodiment, liposomal BoNT is prepared by a dehydration-rehydration method. Formulation of potent bacterial toxins into liposomes requires a meticulous approach. BoNT cannot be exposed to organic solvents that are generally used in manufacture of liposomes. In a preferred method, liposomes encapsulating BoNT are prepared using a thin film hydration method and the lipid dipalmitoyl phosphatidylcholine (DPPC). Briefly, a solution of DPPC in chloroform is first evaporated under a thin stream of nitrogen in a round bottom flask. The lipid film is dried overnight under vacuum. Dried lipids are then hydrated with aqueous BoNT solution or suspension.

After liposomes are prepared, the liposomes are hydrated with a solution of BoNT in water for injection having a suitable concentration, such as 50 units/ml, at 37° C. Then the mixture is incubated at the temperature of 37° C. for a suitable period of time to form oligolamellar hydration liposomes. For example, in a bench scale set up, the mixture was incubated using a water bath for 2 hours.

A cryoprotectant, such as mannitol, is added to the mixture at a suitable concentration, such as 0.5%, 1%, 2.5% or 5% cryoprotectant (w/v) prior to freezing. For example, mannitol may be added to the final mixture at a concentration of 0.5%, 1%, 2.5% or 5% mannitol (w/v), before freezing in acetone-dry ice bath Mannitol acts as a cryoprotectant in the freeze-drying process. The frozen mixture is lyophilized for a suitable period of time, such as at −40° C. and 5 millibar overnight.

The lyophilized cake is then resuspended with saline to the desired final concentration of BoNT. The free BoNT is removed from entrapped BoNT by a suitable method, such as centrifugation at 12,000×g for 30 min using ultracentrifuge. After washing, the precipitates are again resuspended in saline, PBS, or another pharmaceutically acceptable carrier.

The formulations can be stored as liquid, gels and solids. In one embodiment, the formulations are frozen or refrigerated during storage to extend shelf-life. In one embodiment, the liposomes are provided in the form of a dry, powder containing dehydrated BoNT encapsulated liposomes. For example, the BoNT encapsulated liposomes can be provided in a dry (e.g. freeze-dried) form, and be reconstituted with an aqueous solution immediately prior to administration. Shortly before use, for example, within two hours, the dry powder is reconstituted in a pharmaceutically acceptable aqueous carrier. The BoNT encapsulated liposomes can be hydrated by dispersing the liposomes in an aqueous solution with vigorous mixing.

### III. Treatment of Gastric Disorders or Symptoms Thereof With the Liposomal BoNT Formulations

A. Administration

The formulations can be administered to various areas of the GI tracts, such as the stomach, esophagus, small intestine and large intestine, preferably by using endoscopy or panendoscopy and an applicator suitable to administer the formulation, including, but not limited to, a spraying device, gauze, roller or sponge. The formulations can be administered by spraying, painting, rolling or sponging, preferably by spraying using a spraying device.

The formulations containing liposomal BoNT can be administered to a desired location in the gastrointestinal tract by spraying, rolling, painting or sponging a liquid, viscous liquid or gel-like material using an endoscope. The endoscope allows one to identify the area of administration before administering the formulation. The endoscope can include an applicator for the formulation including, but not limited to, a spraying device, gauze, roller or sponge containing the formulation. The applicator can be protected using a suitable cover until the formulation is to be administered so the formulation is not accidentally applied to an undesired area. The applicator can be attached at the end of endoscope to allow high precision administration. Liquid spray tool for endoscope are known in the art, for example such tool is described in U.S. Pat. Nos. 7,588,172 and 6,354,519 to Yamamoto and Kidooka.

The formulations containing liposomal BoNT can be sprayed in a suitable amount and concentration to the site in the GI tract in need of treatment. The formulations containing liposomal BoNT can be painted on the surface of the selected area in the GI tract to coat the surface with the formulation, preferably a viscous formulation or gel-like formulation.

B. Dosage

One advantage with liposomal BoNT delivery is the ability to decrease dosage compared to the dosage required when administering a formulation of unencapsulated BoNT, while achieving the same therapeutic effect. The liposomes enhance the delivery of BoNT resulting in the effectiveness of lower dosages. The liposomal BoNT delivery also increases the effectiveness of treatment for a specified dosage of BoNT. For example, liposomal delivery of BoNT produces a more effective treatment compared to injection of unencapsulated BoNT. As described in Kroupa, et al., Dis Esophagus., 23(2):100-5 (2010), each patient received injection of 200 IU of BoNT into the lower esophageal sphincter (LES) during endoscopy. Administration of high dosages of BoNT is not desirable as BoNT is a potent toxin that can cause paralysis.

The dose of BoNT in the liposomal formulations is lower than the dose required for injected, unencapsulated BoNT required to achieve the same effect. In one embodiment, the dose of BoNT is from about 1 to about 200 units, preferably from about 1 to about 50 units, more preferably from about 1 to about 25 units, and most preferably from about 1 to about 10 units.

In one embodiment, the dose of BoNT is from about 1 to about 100 units. In a more preferred embodiment, the dose of BoNT is from about 1 to about 50 units. In a more preferred embodiment the dose of BoNT is from about 1 to about 25 units. In a most preferred embodiment the dose of BoNT is from about 1 to about 10 units.

Different size dosage units may be used. A dosage unit containing a dry powder of the dehydrated liposomal BoNT can be reconstituted in a container with a pharmaceutically acceptable carrier, preferably a pharmaceutically acceptable aqueous carrier. Suitable amounts include, but are not limited to, 0.1-1 mg, 1-3 mg, 3-10 mg, 10-20 mg and 20-50 mg. Suitable concentrations include, but are not limited to, 0.05 mg/ml to 10 mg/ml, preferably 0.05 mg/ml to 5 mg/ml, more preferably 0.05 mg/ml to 2.5 mg/ml.

The volume of formulation containing the liposome-BoNT is important in the efficacy of delivery. Routine experimentation can be used to determine the delivery volume.

The dosage formulation can be a single dose formulation or a multiple dose formulation. The dosage formulation can come in a single container with a divider between the carrier and the dry powder. The divider can be removed and the dosage formulation can be created by mixing the carrier and dry powder. The dosage formulation can be stored to increase the shelf life of the formulation, for example in a freezer or refrigerator.

A single administration can be effective for more than one week, preferably more than two weeks, more preferably more than three weeks following administration.

C. Disorders and Symptoms to be Treated

The relief from a gastric disorder or one or more symptoms thereof can be greater than a week, a few weeks, one, two or three two months, preferably greater than 6 months, following administration of the formulation where the relief does not decline for a prolonged period of time relative to the current therapies. The formulation can be administered with such regularity to provide effective relief from one or more gastric disorder or symptoms associated with gastric disorders.

The formulation containing liposomal BoNT is administered to a patient with one or more gastric disorders in a sufficient dose to alleviate the gastric disorder or one or more symptoms of the gastric disorder. Improved efficacy in treatment of gastric disorders with botulinum toxin is obtained using liposomal encapsulated botulinum formulations for administration of the botulinum toxin. The liposomes are typically administered in a physiologically acceptable carrier such as saline or phosphate buffered saline by instillation into the gastrointestinal (GI) tract. Representative gastric disorders that can be treated with the formulations include, but are not limited to, gastroesophageal reflux disease (GERD), achalasia, Crohn's disease, diverticulosis, diverticulitis, gallstones, hiatal hernia, gastric stasis, pyloric valve (or other G1 sphincter) malfunction or spasm, H. pylori induced ulcers, peptic ulcers, esophageal spasm, irritable bowel syndrome, stomach ulcers, duodenal ulcers, colitis and ulcerative colitis. In one embodiment, the gastric disorder is GERD, achalasia or an ulcer. In a preferred embodiment, the gastric disorder is GERD. Other disorders to be treated include cancer, infection, spasticity, pain and inflammation.

The formulation containing liposomal BoNT is administered to a patient with a gastric disorder in a sufficient dose to alleviate the gastric disorder or one or more symptoms of the gastric disorder. Symptoms that may be alleviated following administration of the formulation include, but are not limited to, heartburn, belching, regurgitation of food, nausea, vomiting, hoarseness of voice, sore throat, difficulty swallowing, chest pain, and cough.

Representative locations for administration of the formulation to the GI tract include, but are not limited to, the esophagus, stomach, small intestine and large intestine. In one embodiment, the location for administration of the formulation to the GI tract is the pylorus or the esophagus, such as the lower esophageal sphincter or upper esophageal sphincter.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A method of treating a disorder of an organ of the gastrointestinal tract comprising topically administering to the organ of the gastrointestinal tract an effective amount of a composition comprising a lipid vehicle and a botulinum toxin to treat a disorder of the gastrointestinal tract or to alleviate one or more symptoms of the disorder.

2. The method of claim 1, wherein the lipid vehicle is selected from the group consisting of a micelle, an emulsion and a liposome.

3. The method of claim 1, wherein the lipid vehicle is a liposome.

4. The method of claim 3, wherein the liposome comprises a lipid selected from the group consisting of a phospholipid, a glycolipid, a sphingolipid, sphingophospholipid, and a sphingoglycolipid.

5. The method of claim 4, wherein the lipid is selected from the group consisting of phosphatidylcholine, phosphatidylethanolamine, phosphatidylserine, phosphatidylinositol, phosphatidylglycerol, cardiolipin, glycolipids, sphingomyelin, sphingosine I-phosphate, ceramide galactopyranoside, gangliosides, cerebroside, cholesterol, 1,2-distearoylsn-glycero-3-phosphocholine, 1,2-dioleoylphosphatidylcholine and combinations thereof.

6. The method of claim 3, wherein the liposomes comprise one or more lipids, and wherein the ratio of botulinum toxin to lipid ranges from 1:1 to 1:0.1.

7. The method of claim 4, wherein the liposomes comprise sphingomyelin.

8. The method of claim 1, wherein the formulation further comprises a pharmaceutically acceptable aqueous carrier.

9. The method of claim 1, wherein the formulation is administered as a spray.

10. The method of claim 1, wherein the formulation is administered via an endoscope comprising an applicator selected from the group consisting of a spray device, gauze, roller, and sponge.

11. The method of claim 1, wherein the organ of the gastrointestinal tract is the esophagus.

12. The method of claim 1, wherein the disorder of the gastrointestinal tract is selected from the group consisting of cancer, infection, spasticity, pain and inflammation.

13. The method of claim 1, wherein the disorder of the gastrointestinal tract is selected from the group consisting of gastroesophageal reflux disease (GERD), achalasia, Crohn's disease, diverticulosis, diverticulitis, gallstones, hiatal hernia, gastric stasis, pyloric valve (or other GI sphincter) malfunction or spasm, H. pylori induced ulcers, peptic ulcers, esophageal spasm, irritable bowel syndrome, stomach ulcers, duodenal ulcers, colitis and ulcerative colitis.

14. The method of claim 1 wherein an effective amount of the composition is administered to alleviate one or more symptoms selected from the group consisting of heartburn, belching, regurgitation of food, nausea, vomiting, hoarseness of voice, sore throat, difficulty swallowing, chest pain, and cough.

15. The method of claim 1, wherein the botulinum toxin is selected from the group consisting of botulinum toxin A, botulinum toxin B, botulinum toxin C, botulinum toxin D, botulinum toxin E, botulinum toxin F and botulinum toxin G.

16. A pharmaceutical composition comprising a lipid vehicle and a botulinum toxin, wherein the pharmaceutical composition is suitable for topical administration to the eosophagus using an endoscope.

17. The pharmaceutical composition of claim 16 wherein the pharmaceutical composition is a suspension, gel or dry powder.

18. The pharmaceutical composition of claim 16, wherein the botulinum toxin is selected from the group consisting of botulinum toxin A, botulinum toxin B, botulinum toxin C, botulinum toxin D, botulinum toxin E, botulinum toxin F and botulinum toxin G.

19. The pharmaceutical composition of claim 16, wherein the lipid vehicle is selected from the group consisting of a micelle, an emulsion and a liposome.

20. The pharmaceutical composition of claim 16, wherein the lipid vehicle is a liposome.

21. The pharmaceutical composition of claim 20, wherein the liposome comprises lipid selected from a phospholipid, a glycolipid, a sphingolipid, sphingophospholipid, and a sphingoglycolipid.

22. The pharmaceutical composition of claim 21, wherein the lipid is selected from the group consisting of phosphatidylcholine, phosphatidylethanolamine, phosphatidylserine, phosphatidylinositol, phosphatidylglycerol, cardiolipin, glycolipids, sphingomyelin, sphingosine I-phosphate, ceramide galactopyranoside, gangliosides, cerebroside, cholesterol, 1,2-distearoylsn-glycero-3-phosphocholine, 1,2-dioleoylphosphatidylcholine and combinations thereof.

23. The method of claim 20, wherein the liposome comprises one or more lipids, and wherein the ratio of botulinum toxin to lipid ranges from 1:1 to 1:0.1.

24. A dosage formulation of a dry powder which is reconstituted with a pharmaceutically acceptable aqueous carrier for topical administration to the gastrointestinal tract, wherein the dosage formulation comprises
(a) a container of between 0.1 and 1 mg, between greater than 1 and 3 mg, between greater than 3 and 10 mg and between greater than 10 and 20 mg of dry powdered liposomes having botulinum toxin encapsulated therein, and
(b) a container comprising a pharmaceutically acceptable diluent for the liposomes,
wherein, upon reconstitution of the dry powder in the container with the pharmaceutically acceptable diluent suitable for spraying, a liposomal-botulinum mass concentration between 0.05 mg/ml to 10 mg/ml in solution is formed.
